(19) United States
(12) Patent Application Publication
(10) Pub. No.: US 2017/0003284 A1
(43) Pub. Date: Jan. 5, 2017

(54) Western blot process validation and quantification system

(71) Applicant: Alpha Diagnostic International, Inc., San Antonio, TX (US)

(72) Inventor: Masarrat Ali, San Antonio, TX (US)

(21) Appl. No.: 15/197,087

(22) Filed: Jun. 29, 2016

Related U.S. Application Data
(60) Provisional application No. 62/186,637, filed on Jun. 30, 2015.

(51) Int. Cl. G01N 33/543

(52) U.S. Cl. CPC G01N 33/54386

(57) ABSTRACT

Provided herein are strip-based validation and quantification systems, and methods of use thereof, for use in troubleshooting Western blot procedures. The strips and methods disclosed herein also provide a means of absolute quantification of a protein of interest in a Western blot.

### CROSS-REFERENCE TO RELATED APPLICATIONS

This application claims the benefit of U.S. Provisional Application No. 62/186,637, filed Jun. 30, 2015, the disclosure of which is hereby incorporated by reference in its entirety.

### FIELD OF THE INVENTION

This invention relates to the design and development of a novel strip-based validation and quantification system for use in Western blot analytical methods.

### BACKGROUND

The Western blot (protein immunoblot) is a widely used analytical technique for detection of specific protein(s) within complex biological mixtures (e.g., blood, tissue homogenates, tissue extracts). The basic procedure involves fractionating complex mixtures of proteins from multiple samples simultaneously on a matrix such as polyacrylamide gel, followed by transfer of the fractionated proteins onto a membrane made typically of nitrocellulose, nylon, or PVDF. Next, the membrane is sequentially treated with a primary antibody that will specifically bind to a protein of interest, then a secondary antibody that binds to and enables identification of the primary antibody. Detection of bound secondary antibody is typically accomplished via methods such as radioactivity, fluorescence, chemiluminescence, or visible color using a moiety that was previously conjugated to the secondary antibody.

The large number of steps involved from start to finish in the Western blot process, the potential for a variety of human and non-human errors within each step (e.g., failure to include any primary or secondary antibody, using the wrong primary or secondary antibody, using the primary in place of the secondary antibody, reagents that are or have gone bad, or have reduced efficacy etc.), combined with the fact that any processing error will not be evident until the final detection step, all make it very difficult to troubleshoot any unexpected experimental results.

Furthermore, the inherent variability in sample protein concentrations, along with potential gel-to-membrane transfer issues, make quantification of the specific protein of interest quite challenging. At best, the Western blot method can only yield relative or semi-quantitative measure of protein concentrations within and between samples.

The most common approach for obtaining quantitative measure of the protein of interest in a given sample is to measure the amount of protein such as tubulin or beta-actin in the same sample and compare the relative amount of the protein of interest to the amount of tubulin or beta-actin. The amount of beta-actin or tubulin between samples is believed to be relatively steady and therefore, it can be used to normalize the total protein concentrations between samples. However, this method only yields relative measure of protein amount within a sample and not absolute quantity.

In summary, there are no simple and reliable methods by which one can troubleshoot unexpected experimental results in a Western blot experiment. Moreover, techniques enabling one to quantify the amount of specific proteins in a Western blot accurately do not exist. There is thus a need in the art for a means to troubleshoot results in Western blot experiments. Such a system would also enable quantification of one or more proteins of interest.

### SUMMARY

The methods and devices disclosed herein are not limited to specific advantages or functionality.

In one aspect, the disclosure provides Western blot process validation and quantification strips comprising a strip body, which comprises: (a) a primary-antibody validation zone comprising a primary-antibody validation reagent, wherein the primary-antibody validation reagent binds specifically to a Western blot primary antibody; (b) a secondary-antibody validation zone comprising a secondary-antibody validation reagent, wherein the secondary-antibody validation reagent binds specifically to a Western blot secondary antibody; and (c) a detection validation zone comprising a detection reagent, wherein the detection reagent reacts with a detection substrate. In some embodiments, the strip body comprises a nitrocellulose or PVDF membrane.

In some embodiments, the primary-antibody validation reagent is an anti-rabbit antibody, an anti-goat antibody, an anti-sheep antibody, an anti-mouse antibody, or an anti-rat antibody; and wherein the primary-antibody validation reagent is isolated from an organism different from that of the Western blot primary antibody. In some embodiments, the secondary-antibody validation reagent is isolated from the same organism as that of the Western blot primary antibody. In some embodiments, the detection validation zone comprises two detection reagents, which are horseradish peroxidase and alkaline phosphatase.

In some embodiments, the primary-antibody validation zone and secondary-antibody validation zone each comprise at least two different amounts of the validation reagent therein.

In some embodiments, the Western blot process validation and quantification strips further comprise a quantification zone comprising at least two different amounts of a quantification reagent. In some embodiments, the quantification reagent is a housekeeping protein selected from the group consisting of GAPDH, beta-actin, and beta-tubulin. In some embodiments, the quantification reagent is the same protein as the target protein of interest that is being investigated via the Western blot analytical method. For example, if the target protein of interest in the sample being investigated is the p53 tumor antigen, the quantification reagent may be p53 rather than a housekeeping protein, such as actin, tubulin or GAPDH.

In some embodiments, the reagents in each zone are laid as stripes in adjacent lines across the width of the strip.

In another aspect, the disclosure provides methods of validating a Western blot experiment, the methods comprising co-processing the Western blot process validation strip according to the disclosure along with a Western blot membrane, wherein the Western blot experiment is validated where colored deposits are detected in the primary-antibody validation zone, the secondary-antibody validation zone, and the detection validation zone of the validation strip.

In another aspect, the disclosure provides methods of quantifying the amount of housekeeping protein in a Western blot, the methods comprising: (a) incubating a validation and quantification strip according to the disclosure and a Western blot membrane with an antibody specific for the quantification reagent, wherein the quantification reagent is a housekeeping protein; (b) processing the validation strip and the Western blot membrane to enable detection of the quantification reagent; (c) determining the intensities of the at least two different amounts of quantification reagent in the quantification zone, and determining the intensities of the bands corresponding to the housekeeping protein in the Western blot; (d) generating a standard curve correlating the intensities of the quantification reagent in the quantification zone to the amounts of quantification reagent in the quantification zone; (e) quantifying the amount of the housekeeping protein in each lane of the Western blot by correlating the housekeeping protein intensities to housekeeping protein amounts using the standard curve.

In some embodiments, the methods further comprise: in step (c), normalizing the intensities of the bands corresponding to the housekeeping protein to a single housekeeping protein band intensity; or, in step (e), normalizing the amounts of housekeeping protein in each lane of the Western blot to a single amount of housekeeping protein. In some embodiments, the single housekeeping protein band intensity is the intensity of the highest-intensity housekeeping protein band on the Western blot, and wherein the single amount of housekeeping protein is the highest amount of housekeeping protein in any one lane on the Western blot. In some embodiments, the single housekeeping protein band intensity is the average of the intensities of the housekeeping proteins bands on the Western blot, and wherein the single amount of housekeeping protein is the average of the amounts of housekeeping protein from all lanes on the Western blot.

In another aspect, the disclosure provides methods of quantifying the amount of a target protein of interest in a Western blot, the methods comprising: (a) incubating a validation and quantification strip according to the disclosure and a Western blot membrane with an antibody specific for the quantification reagent, wherein the quantification reagent is the target protein of interest being investigated in the sample; (b) processing the validation strip and the Western blot membrane to enable detection of the quantification reagent in the validation strip, and to enable detection of the target protein of interest in the Western blot; (c) determining the intensities of the at least two different amounts of quantification reagent in the quantification zone, and determining the intensities of the bands corresponding to the target protein of interest in the Western blot; (d) generating a standard curve correlating the intensities of the quantification reagent in the quantification zone to the amounts of quantification reagent in the quantification zone; (e) using the standard curve to determine the amount of target protein of interest in each lane from previously determined intensities of protein of interest in each lane.

In another aspect, the disclosure provides methods of quantifying the amount of a protein of interest in a Western blot, the methods comprising: (a) incubating a validation and quantification strip according to the disclosure and a Western blot membrane with an antibody specific for a quantification reagent, wherein the quantification reagent is a housekeeping protein; (b) processing the validation strip and the Western blot to enable detection of the quantification reagent; (c) determining the intensities of the at least two different amounts of quantification reagent in the quantification zone, and determining the intensities of the bands corresponding to the housekeeping protein in the Western blot; (d) generating a standard curve correlating the intensities of the quantification reagent in the quantification zone to the amounts of quantification reagent in the quantification zone; and (e) using the standard curve to determine the amount of protein of interest in each lane from previously determined intensities of protein of interest in each lane.

In some embodiments, the methods further comprise using a normalization factor for each lane to normalize the previously determined intensities of protein of interest in each lane or to normalize the amounts of protein of interest in each lane, wherein the normalization factor for a given lane is determined by calculating the factor necessary to normalize the intensity of the housekeeping protein band in that lane to a single housekeeping protein band intensity, or the factor necessary to normalize the amount of housekeeping protein in that lane to a single amount of housekeeping protein. In some embodiments, the single housekeeping protein band intensity is the intensity of the highest-intensity housekeeping protein band on the Western blot, and wherein the single amount of housekeeping protein is the highest amount of housekeeping protein in any one lane on the Western blot. In some embodiments, the single housekeeping protein band intensity is the average of the intensities of the housekeeping proteins bands on the Western blot, and wherein the single amount of housekeeping protein is the average of the amounts of housekeeping protein from all lanes on the Western blot.

These as well as other aspects, advantages, and alternatives, will become apparent to those of ordinary skill in the art by reading the following detailed description, with reference where appropriate to the accompanying drawings, and taken together with the accompanying claims.

### BRIEF DESCRIPTION OF THE DRAWINGS

An exemplary embodiment of the present invention is described herein with reference to the drawings, which are briefly described as follows.

FIG. 1 shows an exemplary validation and quantification strip according to the disclosure with a primary antibody validation zone and reagents (labeled “Zone 2”).

FIG. 2 shows an exemplary validation and quantification strip according to the disclosure as in FIG. 1, but with a secondary antibody validation zone and reagents (labeled “Zone 3”) in addition to the primary antibody validation zone and reagents.

FIG. 3 shows an exemplary validation and quantification strip according to the disclosure as in FIG. 2, but with a detection validation zone and reagents (labeled “Zone 4”) in addition to primary and secondary antibody validation zones and reagents.

FIG. 4 shows an exemplary validation and quantification strip according to the disclosure as in FIG. 3, but with a quantification zone and reagents (labeled “Zone 1”) in addition to the primary and secondary antibody validation zones and the detection validation zone.

FIG. 5A shows an example of results on a validation and quantification strip of the disclosure (left) along with the resulting Western blot (right) when the primary antibody reaction step was unsuccessful; FIG. 5B shows an example of results on a validation and quantification strip of the disclosure (left) along with the resulting Western blot (right) when the all of the reagents and steps were successfully performed and completed as expected.

FIG. 6 shows an example of results on a validation and quantification strip of the disclosure (left) along with the resulting Western blot (right) when tested with a control protein for the quantification step.

FIG. 7 shows a flow chart illustrating the use of a validation and quantification strip of the disclosure for performing validation of a Western blot experiment.

FIG. 8 shows a flow chart illustrating the use of a validation and quantification strip of the disclosure for performing quantification of a protein of interest during a Western blot experiment.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION

All publications, patents and patent applications cited herein are hereby expressly incorporated by reference for all purposes.

Provided herein are strip-based validation and quantification systems that can be used alongside a standard Western blot experimental method in biological applications. The systems disclosed herein comprise strips made from the same types of membrane used in Western blot procedures, such as, for example, nitrocellulose, nylon, or PVDF. The strips are co-processed along with Western blot membranes and are thus taken through the same experimental steps. By comprising reagents and the means for detecting whether or not certain key Western blot process steps have been performed correctly with the correct reagents, the strips provide a way to identify process-related errors to aid in troubleshooting a failed Western blot experiment or unexpected Western blot results. The strips and methods of use thereof disclosed herein also provide a means of absolute quantification of a protein of interest in a Western blot procedure.

Before describing the disclosed methods and devices in detail, a number of terms will be defined. As used herein, the singular forms “a”, “an”, and “the” include plural referents unless the context clearly dictates otherwise. For example, reference to a “protein” means one or more proteins.

It is noted that terms like “preferably,” “commonly,” and “typically” are not utilized herein to limit the scope of the claimed aspects and embodiments or to imply that certain features are critical, essential, or even important to the structure or function of the claimed aspects and embodiments. Rather, these terms are merely intended to highlight alternative or additional features that can or cannot be utilized in a particular embodiment.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Exemplary embodiments are described herein. It should be understood that the word “exemplary” is used herein to mean “serving as an example, instance, or illustration.” Any embodiment described herein as “exemplary” is not necessarily to be construed as preferred or advantageous over other embodiments. Further, those skilled in the art will understand that changes and modifications may be made to these embodiments without departing from the true scope and spirit of the invention, which is defined by the claims.

In one aspect, the disclosure provides Western blot process validation and/or quantification strips comprising a strip body, which comprises: (a) a primary-antibody validation zone comprising a primary-antibody validation reagent, wherein the primary-antibody validation reagent binds specifically to a Western blot primary antibody; (b) a secondary-antibody validation zone comprising a secondary-antibody validation reagent, wherein the secondary-antibody validation reagent binds specifically to a Western blot secondary antibody; and (c) a detection validation zone comprising a detection reagent, wherein the detection reagent reacts with a detection substrate.

In some embodiments, the strip body comprises any material that Western blot membranes can be made from. In some embodiments, the strip body comprises a membrane comprising nitrocellulose, nylon, PVDF, or any other material suitable for performing Western blot.

The validation and/or quantification strips disclosed herein (“V&Q strips”) can be made in a variety of different lengths and widths, for example, to match the length of common Western blot membrane transfer systems that are commercially available. For example, if the Western blot experiment is being performed using a mini-gel system, then the V&Q strips could be 3″ in length. If on the other hand, if the Western blot experiment is being performed using a standard large-gel system, then the V&Q strips could be 6″ in length. In some embodiments, the V&Q strips are from about 1″ (1 inch) to about 10″ (10 inches) in length, or from about 2″ to about 8″ in length, or from about 3″ to about 6″ in length. In some embodiments, the V&Q strips are about 1″, or about 2″, or about 3″, or about 4″, or about 5″, or about 6″, or about 7″, or about 8″, or about 9″, or about 10″ in length. In some embodiments, the V&Q strips are about 3″ in length. In some embodiments, the V&Q strips are about 6″ in length. In some embodiments, the V&Q strips are from about 0.1″ to about 1″, or about 0.2″ to about 0.8″, or from about 0.3″ to about 0.7″, or from about 0.4″ to about 0.6″ in width. In some embodiments, the V&Q strips are about 0.1″, or about 0.2″, or about 0.3″, or about 0.4″, or about 0.5″, or about 0.6″ or about 0.7″ or about 0.8″, or about 0.9″ or about 1″ in width. In some embodiments, the V&Q strips are about 0.5″ in width. In some embodiments, the dimensions of the V&Q strips are approximately 0.5″ in width and 3″ in length.

In some embodiments, V&Q strips have a plurality of zones with each zone comprising specific reagents applied as stripes applied in adjacent lines across the width of the strip. In other embodiments, reagents are applied as spots on the strip. The plurality of zones comprise one or more of a quantification zone, a primary antibody validation zone, a secondary antibody validation zone, or a detection reagent validation zone.

### Primary-Antibody Validation Zone

In the Western blot process validation strips disclosed herein, the primary-antibody validation zone enables a user to determine whether a Western blot primary antibody has been correctly selected and applied during a Western blot procedure. For example, if the user will be performing a Western blot that uses a primary antibody raised in rabbits to detect a protein of interest, the user will add to the Western blot membrane (and to the co-processed validation strip) the primary antibody. To capture and later detect the same primary antibody, the strip will have in the primary-antibody validation zone a rabbit antibody capture reagent, such as an anti-rabbit IgG antibody. Since the strip is processed alongside the Western blot, the rabbit antibody used in the Western blot procedure to detect the protein of interest will also bind to the rabbit antibody capture reagent in the primary-antibody validation zone. If the Western blot, along with the co-processed validation strip, is then processed correctly following addition of the primary antibody, the primary-antibody validation zone will show that the primary antibody addition step was correctly performed.

In some embodiments, the primary-antibody validation reagent is an anti-rabbit antibody, an anti-goat antibody, an anti-sheep antibody, an anti-mouse antibody, or an anti-rat antibody. In some embodiments, the primary-antibody validation reagent is isolated from an organism different from that of the Western blot primary antibody. For example, if the Western blot primary antibody being used by the user was raised in rabbits, and the primary-antibody validation reagent is an anti-rabbit antibody, the primary-antibody validation reagent may have been raised in any animal other than rabbits; for example, it may be a goat anti-rabbit antibody, or a sheep anti-rabbit antibody, a mouse anti-rabbit antibody, etc.

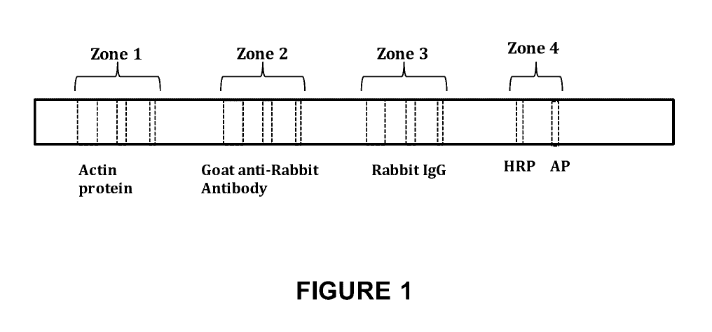

FIG. 1 illustrates an exemplary V&Q strip as disclosed herein with primary antibody validation reagent applied in Zone 2. In the example described above, the experimental Western blot membrane is probed with primary antibodies that have been raised in rabbits. Therefore, the reagent that is applied in zone 2 comprises an antibody that will specifically bind to the rabbit primary antibody. More specifically, the zone 2 reagent in this example comprises an antibody that is raised in goats to specifically detect and capture antibodies that have been raised in rabbits.

In some embodiments, the primary-antibody validation zone comprises at least two different amounts of the primary-antibody validation reagent therein. The different amounts of primary-antibody validation reagent enable a user to determine or troubleshoot whether the correct amount of primary antibody has been added to the Western blot (and to the co-processed V&Q strip). In a typical Western blot, the primary antibody is diluted to a specific concentration in a defined volume of the incubation buffer that has been empirically determined to provide optimal results. This dilution factor depends on the “titer” of the primary antibody reagent. The purpose of having at least two different amounts of the primary antibody validation reagent in zone 2 is thus to determine whether the correct dilution of the primary antibody was used in the experiment. Take, for example, a primary-antibody validation zone comprising three different amounts of primary-antibody validation reagent. These different amounts of primary antibody validation reagent can be designed such that only the middle and the highest concentration stripes of the primary antibody validation reagents would show positive results at the conclusion of an experiment where the correct dilution of primary antibody was applied to the Western blot. In such a design, if the primary antibody is dilution is much higher, only the highest concentration of the validation reagent stripe would be detectable. Likewise, if the dilution of the primary antibody is too low, then all three concentrations of the validation reagent stripes would be detectable. In this fashion, it is possible to validate the correct dilution of the primary antibody in the incubation buffer.

Following these principles, the primary-antibody validation zone can comprise, in some embodiments, one, or two, or three, or four, or five, or six, or seven, or eight, or nine, or ten different predetermined amounts of primary-antibody validation reagent. In some embodiments wherein there are two different amounts of primary-antibody validation reagent in the primary-antibody validation zone, the amounts are designed such that only the reagent stripe with the higher amount is detectable when the correct dilution of primary antibody is used. In some embodiments wherein there are two different amounts of primary-antibody validation reagent in the primary-antibody validation zone, the amounts are designed such that only the reagent stripe with the lower amount is detectable when the correct dilution of primary antibody is used.

In some embodiments wherein there are more than two different amounts of primary-antibody validation reagent in the primary-antibody validation zone, the amounts are designed such that only the reagent stripe or stripes with the middle amount(s) are detectable when the correct dilution of primary antibody is used. In some embodiments wherein there are more than two different amounts of primary-antibody validation reagent in the primary-antibody validation zone, the amounts are designed such that only the reagent stripe or stripes with the lower amount(s) are detectable when the correct dilution of primary antibody is used. In some embodiments wherein there are more than two different amounts of primary-antibody validation reagent in the primary-antibody validation zone, the amounts are designed such that only the reagent stripe or stripes with the higher amount(s) are detectable when the correct dilution of primary antibody is used.

In some embodiments, each of the different predetermined amounts of primary-antibody validation reagent in the primary-antibody validation zone is accurate to within from about 0.1% to about 10% of that particular amount of primary-antibody validation reagent, or from about 0.5% to about 5% of that amount. In some embodiments, the amount is accurate to within about 5%, or about 4%, or about 3%, or about 2%, or about 1%, or about 0.5%, or about 0.1% of the amount. In some embodiments, each of the different predetermined amounts of primary-antibody validation reagent is accurate to within about 0.001 mg to about 100 mg, or to within about 0.01 mg to about 10 mg, or to within about 0.01 mg to about 1 mg. In some embodiments, each of the different amounts of primary-antibody validation reagent in the primary-antibody validation zone is accurate to within 0.01 mg or to within 0.1 mg.

### Secondary-Antibody Validation Zone

In the Western blot process validation strips disclosed herein, the secondary-antibody validation zone enables a user to determine whether a Western blot secondary antibody has been correctly selected and applied during a Western blot procedure. For example, in some embodiments, the secondary-antibody validation zone contains a reagent that will bind to the secondary antibody used in the Western blot. Continuing the same example as above, after addition of the primary antibody raised in rabbits, the user will next add an anti-rabbit secondary antibody to the Western blot in order to detect the primary antibody already bound to the Western blot membrane. To capture and later detect the same secondary antibody, the strip will have in the secondary-antibody validation zone a reagent such as a rabbit IgG (immunoglobulin G) capable of binding to and capturing the secondary antibody. Thus, if all steps have been performed correctly, the strip up to this point will have (1) primary antibody bound to the primary-antibody validation zone and (2) secondary antibody bound to both the secondary-antibody validation zone and the primary-antibody validation zone (via the previously captured primary antibody).

In some embodiments, the secondary-antibody validation reagent is isolated from the same organism as that of the Western blot primary antibody. For example, if a rabbit primary antibody is being used in the Western blot procedure, the secondary antibody used in the Western blot procedure will be an anti-rabbit antibody, and the secondary-antibody validation reagent may be any protein, such as an affinity-purified immunoglobulin G protein, which was isolated from rabbits. In this manner, the secondary antibody will bind to the secondary-antibody capture reagent.

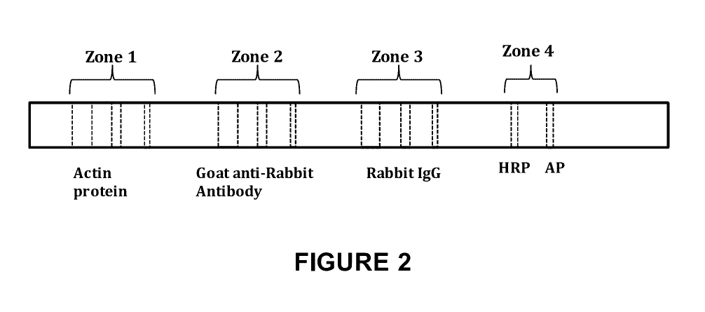

FIG. 2 illustrates the exemplary V&Q strip from FIG. 1 with secondary antibody validation reagent applied in zone 3. This is in addition to the primary antibody validation reagent that is already present in zone 2. In this example, the experimental Western blot membrane that was initially incubated with a primary antibody will now be probed with a secondary antibody that will specifically bind to the rabbit primary antibody bound to specific regions in the membrane. Therefore, as described above, a rabbit IgG molecule is applied in zone 3, which will specifically bind to and capture the secondary antibody. Similar to zone 2, the secondary antibody validation reagent is, in this example, applied in several different concentrations in zone 3.

In some embodiments, the secondary-antibody validation zone comprises at least two different amounts of the secondary-antibody validation reagent therein. The different amounts of secondary-antibody validation reagent enable a user to determine or troubleshoot whether the correct amount of secondary antibody has been added to the Western blot (and to the co-processed V&Q strip). In a typical Western blot, the secondary antibody is diluted to a specific concentration in a defined volume of the incubation buffer that has been empirically determined to provide optimal results. This dilution factor depends on the “titer” of the secondary antibody reagent. The purpose of having at least two different amounts of the secondary antibody validation reagent in zone 2 is thus to determine whether the correct dilution of the secondary antibody was used in the experiment. Take, for example, a secondary-antibody validation zone comprising three different amounts of secondary-antibody validation reagent. These different amounts of secondary antibody validation reagent can be designed such that only the middle and the highest concentration stripes of the secondary antibody validation reagents would show positive results at the conclusion of an experiment where the correct dilution of secondary antibody was applied to the Western blot. In such a design, if the secondary antibody is dilution is much higher, only the highest concentration of the validation reagent stripe would be detectable. Likewise, if the dilution of the secondary antibody is too low, then all three concentrations of the validation reagent stripes would be detectable. In this fashion, it is possible to validate the correct dilution of the secondary antibody in the incubation buffer.

Following these principles, the secondary-antibody validation zone can comprise, in some embodiments, one, or two, or three, or four, or five, or six, or seven, or eight, or nine, or ten different predetermined amounts of secondary-antibody validation reagent. In some embodiments wherein there are two different amounts of secondary-antibody validation reagent in the secondary-antibody validation zone, the amounts are designed such that only the reagent stripe with the higher amount is detectable when the correct dilution of secondary antibody is used. In some embodiments wherein there are two different amounts of secondary-antibody validation reagent in the secondary-antibody validation zone, the amounts are designed such that only the reagent stripe with the lower amount is detectable when the correct dilution of secondary antibody is used.

In some embodiments wherein there are more than two different amounts of secondary-antibody validation reagent in the secondary-antibody validation zone, the amounts are designed such that only the reagent stripe or stripes with the middle amount(s) are detectable when the correct dilution of secondary antibody is used. In some embodiments wherein there are more than two different amounts of secondary-antibody validation reagent in the secondary-antibody validation zone, the amounts are designed such that only the reagent stripe or stripes with the lower amount(s) are detectable when the correct dilution of secondary antibody is used. In some embodiments wherein there are more than two different amounts of secondary-antibody validation reagent in the secondary-antibody validation zone, the amounts are designed such that only the reagent stripe or stripes with the higher amount(s) are detectable when the correct dilution of secondary antibody is used.

In some embodiments, each of the different predetermined amounts of secondary-antibody validation reagent in the secondary-antibody validation zone is accurate to within from about 0.1% to about 10% of that particular amount of secondary-antibody validation reagent, or from about 0.5% to about 5% of that amount. In some embodiments, the predetermined amount is accurate to within about 5%, or about 4%, or about 3%, or about 2%, or about 1%, or about 0.5%, or about 0.1% of the amount. In some embodiments, each of the different amounts of secondary-antibody validation reagent is accurate to within about 0.001 mg to about 100 mg, or to within about 0.01 mg to about 10 mg, or to within about 0.01 mg to about 1 mg. In some embodiments, each of the different predetermined amounts of secondary-antibody validation reagent in the secondary-antibody validation zone is accurate to within 0.01 mg or to within 0.1 mg.

### Detection Validation Zone

In the Western blot process validation strips disclosed herein, the detection validation zone enables a user to determine whether the one or more Western blot detection reagents have been correctly selected and applied during a Western blot procedure.

In some embodiments, the detection validation zone comprises one or more reagents that react with a detection substrate. A later step in a typical Western blot process is to incubate the experimental blot with reagents that will enable detection of the secondary antibodies that are bound to the primary antibodies. Typically, Western blot secondary antibodies are conjugated to enzymes such as horseradish peroxidase (HRP) or alkaline phosphatase (AP). When HRP or AP comes into contact with their specific substrates, provided in the medium around the Western blot membrane, the reaction produces a visible colored deposit at the site of the reaction.

Thus, in some embodiments, the detection validation zone comprises regions comprising one or more enzymes that are typically bound to secondary antibodies in Western blot procedures—e.g., HRP and AP. Other enzymes or reagents contemplated for use in the detection validation zone include glucose oxidase, beta-galactosidase and biotin.

In some embodiments, the detection validation zone contains a plurality of stripes, each containing one of the enzymes typically bound to Western blot secondary antibodies. In some embodiments the detection validation zone comprises two detection reagents, such as HRP and AP, each as separate stripes. The use of such a detection validation zone is illustrated by continuing the example from above, in which the secondary antibody in the Western blot process may have been conjugated with HRP. In such a case, the reagent specific for HRP will form colored deposits at the site of the secondary antibodies in the experimental blot. If all steps have been performed correctly up to this point, the co-processed validation strip will also show formation of colored deposits in all three of the primary-antibody validation zone, the secondary-antibody validation zone, and the detection validation zone. In the detection validation zone, only the HRP stripe (and not the AP stripe) will exhibit the colored deposit.

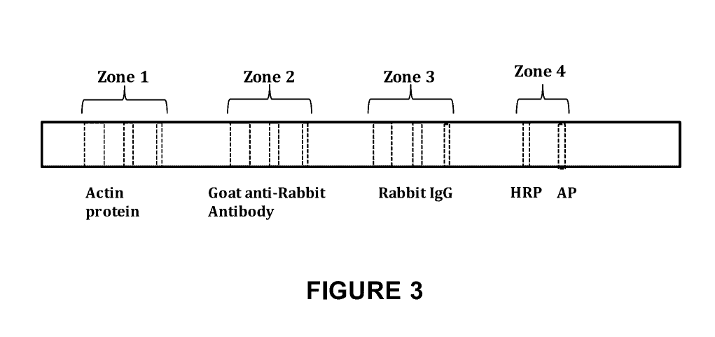

FIG. 3 illustrates the exemplary V&Q strip from FIG. 2 with detection validation reagents applied in zone 4. This is in addition to the primary and secondary antibody validation reagents that are already present in zones 2 and 3, respectively. In the example discussed so far, the detection validation reagents that are applied to the V&Q strip are purified HRP and AP enzymes. Depending on which enzyme is conjugated to the secondary antibody in a particular experiment, the corresponding substrate will interact with the enzyme conjugated to the secondary antibody on the experimental Western blot membrane and the purified enzyme on the V&Q strip to produce the appropriate results.

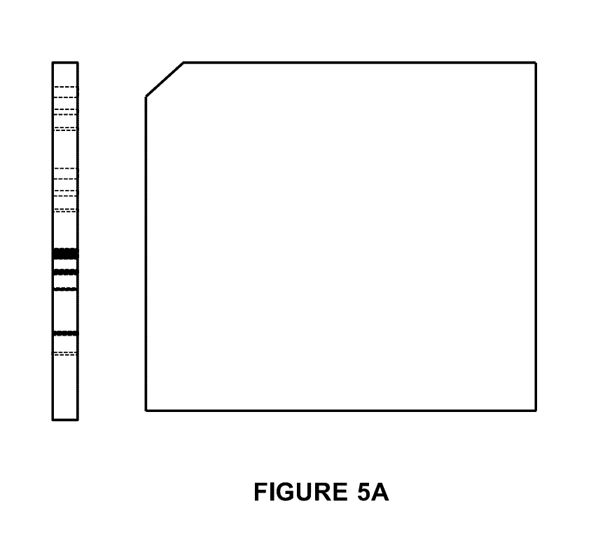

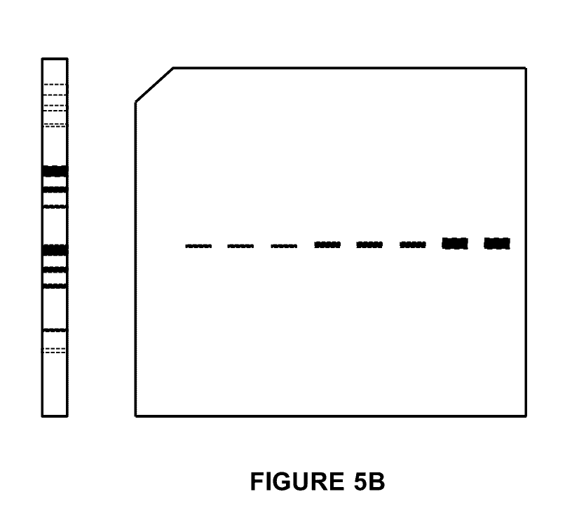

FIGS. 5A and 5B illustrate one such example of how the inclusion of the V&Q strips along with the experimental Western blot can help troubleshoot an unexpected result. FIG. 5A shows an example of an unexpected result where the experimental Western blot membrane provided no visible results after undergoing all the usual steps of the process. In this example, the secondary antibody was conjugated with HRP and therefore the corresponding substrates are used. An observation of the V&Q strips that underwent the exact same steps as the experimental membrane shows that zone 2 (primary antibody validation region) yielded no visible results. This would indicate that the either the primary antibody was accidentally omitted, or an incorrect primary antibody was use, or the primary antibody reagent has gone bad. In any case, it is easy to ascertain that the problem lies with the primary antibody. FIG. 5B shows typical results when all the reagents are functioning normally and the results are as expected with the Western blot membrane. An observation of the corresponding V&Q strips also shows similar results with zones 2, 3, and 4 yielding the expected results. It should be noted that there can be several other instances of unexpected results and in those cases, the inclusion of V&Q strips alongside the experimental Western blots should also enable troubleshooting of unexpected results.

### Quantification Zone

In some embodiments, the Western blot process validation strip further comprises a quantification zone comprising at least two different predetermined amounts of a quantification reagent. The quantification zone enables a user to accurately quantify the amount of one or more proteins of interest in the sample(s) being assayed in the Western blot experiment.

The quantification zone comprises accurately known predetermined amounts of a quantification reagent, which may comprise any known protein. In some embodiments, the expression level of the quantification reagent is not expected to vary from sample to sample. Such proteins are herein referred to as “housekeeping proteins,” examples of which include GAPDH, beta-tubulin, and beta-actin. The expression of these proteins is relatively unchanging between different samples assuming that a constant amount of total protein is used in every sample being fractionated on an experimental gel. Thus, in some embodiments, the quantification reagent is a housekeeping protein. In other embodiments, the quantification reagent may be the same protein as the target protein of interest being investigated using the Western blot, rather than a separate housekeeping protein.

In some embodiments, each predetermined amount of quantification reagent is accurate to within from about 0.1% to about 10% of the predetermined amount of quantification reagent, or from about 0.5% to about 5% of the predetermined amount. In some embodiments, the predetermined amount is accurate to within about 5%, or about 4%, or about 3%, or about 2%, or about 1%, or about 0.5%, or about 0.1% of the predetermined amount. In some embodiments, each predetermined amount of quantification reagent is accurate to within about 0.001 mg to about 100 mg, or to within about 0.01 mg to about 10 mg, or to within about 0.01 mg to about 1 mg. In some embodiments, each predetermined amount of quantification reagent in the quantification zone is accurate to within 0.01 mg or to within 0.1 mg.

In a typical Western blot procedure, by re-probing the membrane with primary and secondary antibody reagents specific for say, actin, and using the resulting actin protein intensities to normalize and correct the experimental blot for any potential sample to sample protein concentration variability, it is possible to obtain a quantitative measure of relative amounts of the target protein of interest compared to the levels of actin in the same sample. Such a method, however, does not provide the actual or absolute amounts of the actin protein or, for that matter, the actual or absolute amount of the target protein within the samples.

The V&Q strips as disclosed herein, on the other hand, enable a user to absolutely quantify the amount of one or more proteins of interest by providing the means by which a user may generate a standard curve that plots intensity of the quantification reagent stripes in the quantification zone versus protein amount (since the stripes of quantification reagent contain known amounts of the reagent). This curve may then be used to correlate the intensity of a protein of interest on the Western blot to a protein amount, thereby allowing absolute quantification of the protein of interest.

In some embodiments, prior to correlating intensity using the standard curve, quantification of a protein of interest also comprises normalizing the intensity of each band corresponding to the protein of interest using a normalization factor particular to the lane on the Western blot in which the band resides. In some embodiments, the lane's normalization factor is determined by calculating a factor necessary to normalize (equalize) the intensity of the housekeeping protein band in that particular lane to the intensities of the housekeeping protein bands in all other lanes. In some embodiments, the housekeeping protein bands are all normalized to the intensity of the housekeeping protein band with the highest intensity. In some embodiments, the housekeeping protein bands are all normalized to the average (mean) intensity of all of the housekeeping protein bands.

Conventionally, and in methods of using the Western blot validation and quantification strips disclosed herein, quantification of a Western blot requires an additional step where the experimental blot is stripped of all reagents, such as primary and secondary antibodies, and the colored or chemiluminescent product deposits, leaving behind only the fractionated proteins that remain on the membrane. After doing so, the blot can be re-incubated with a different primary antibody specific to a housekeeping protein.

Continuing the example from above, after reading the completed Western blot, the blot is then stripped of bound antibodies and/or other reagents and re-processed, starting with incubation with a rabbit anti-actin (or rabbit anti-tubulin, or rabbit anti-GAPDH, etc.) primary antibody. During this incubation, a new validation strip may or may not be included along with the experimental blot (i.e., no need to reuse the previous strip). After this initial incubation with a rabbit anti-actin (or anti-GAPDH, or anti-tubulin) antibody, the experimental blot and the validation strip undergo the same steps as described above. At the end of the experiment, colored deposits should be visible in all 4 zones. The accurately known predetermined amounts, which have now been detected and which appear in the quantification zone then allow production of an accurate protein standard curve for actin that can then be used to quantify the actin in the experimental blot, and in turn to quantify the protein of interest.

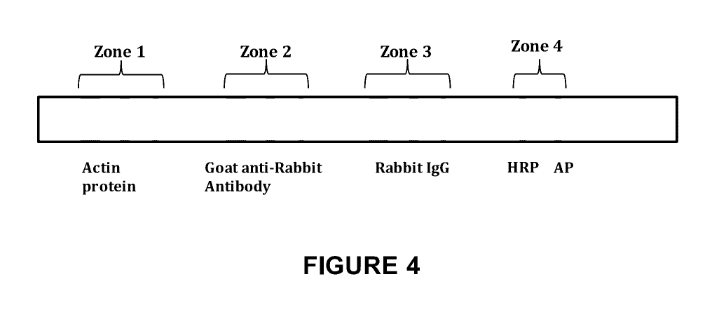

FIG. 4 illustrates the exemplary V&Q strips from the FIGS. 1, 2 and 3 with quantification reagents applied in zone 1. This is in addition to the primary and secondary antibody validation and detection validation reagents that are already present in zones 2, 3 and 4, respectively. In this example, the quantification reagents that are applied to the V&Q strips are purified actin protein. The actin protein is present in 3 different concentrations that enable producing a standard curve that can be used to determine the exact amount of actin protein in any co-processed unknown sample by comparing to the standard curve generated from the actin samples on the V&Q strips.

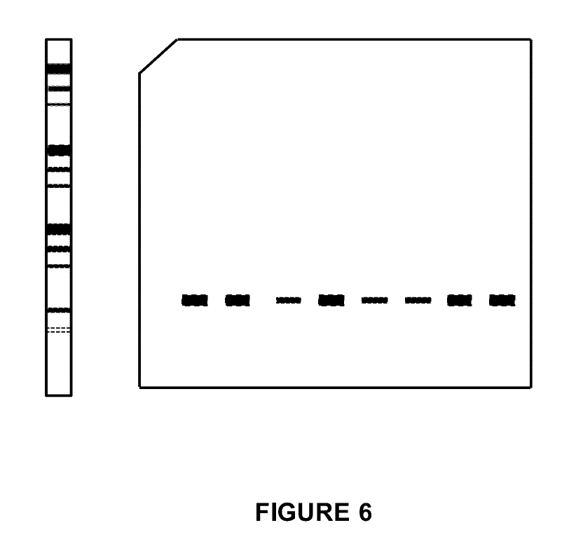

FIG. 6 illustrates an exemplary use of the V&Q strips alongside the experimental Western blot for this specific application. In this example, the housekeeping protein on the strip is actin. The Western blot membrane was stripped of all other reagents and re-probed with a primary and secondary antibody for detection of actin proteins in the different samples. As can be seen, the levels of actin protein in the Western blot membrane are relatively steady, but do vary between samples. To adjust for variations between lanes, the intensity of each actin band is normalized to a single value across the blot so that all actin bands from all lanes have the same intensity. For example, each actin band intensity may be normalized to the intensity of the highest-intensity actin band. Thus, a normalization factor will be determined for each lane, which will be the factor necessary to bring the intensity of the actin band in that lane to be equal to the actin intensities in the other lanes. Optionally, the intensities of the actin band from each sample can be directly compared to the standard curve generated from the intensities of the known amounts of the 3 actin bands in the V&Q strips to determine the exact amount of actin in each band. Once the normalization factor for each lane is determined, it is applied to the intensity of the protein of interest in that lane, yielding normalized intensities for the protein of interest across the blot. These normalized intensities are then converted to actual amounts of protein in each lane using the actin standard curve.

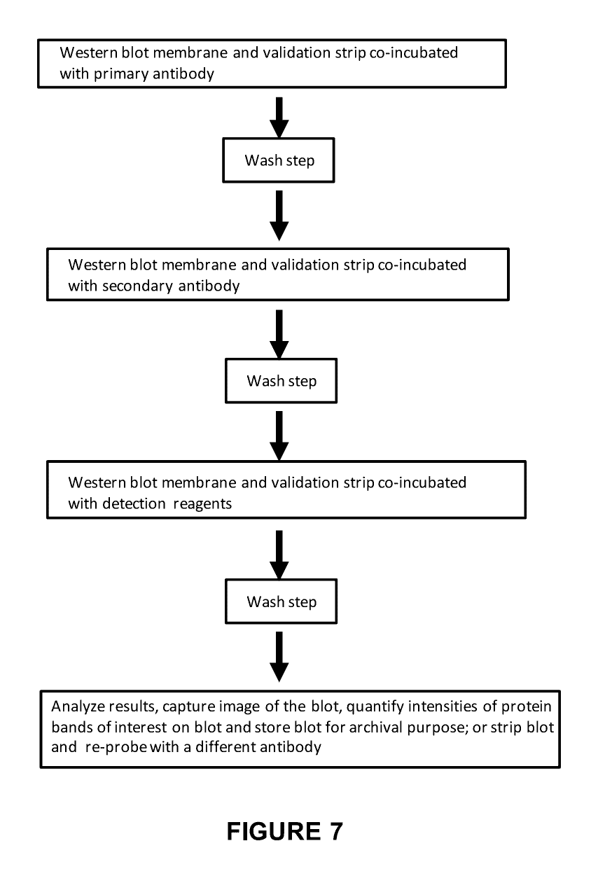

FIG. 7 illustrates a flowchart of typical Western blot process steps that also includes the V&Q strips in the same process. The Western blot membrane containing the immobilized fractionated proteins from the various samples is co-incubated along with a single V&Q strips during the treatment with the primary antibody solution. After a defined amount of time, the primary antibody solution is discarded and the membrane plus V&Q strips are washed thoroughly to remove any primary antibody solution that may have adhered to the membrane and strip. Following this wash step, the membrane and the strip are co-incubated with the secondary antibody solution for a defined amount of time. The secondary antibody solution is discarded and the wash step is performed as above. Lastly, the membrane and strip are treated with the detection reagents corresponding to the enzyme that is conjugated to the secondary antibody and after a defined amount of time, the reagent solution is discarded, washed thoroughly to stop any further chemical reactions and stored for further processing. Preferably, the resulting image from the membrane and the strip is captured digitally and using appropriate quantification software, the intensities of individual protein bands are computed and stored. At this stage, the membrane can either be stored or preferably, stripped of all reagents that are bound to the membrane using special stripping solution, which leaves behind only the original fractionated proteins on the membrane. This then allows the membrane to be re-used to investigate other proteins present within the sample proteins.

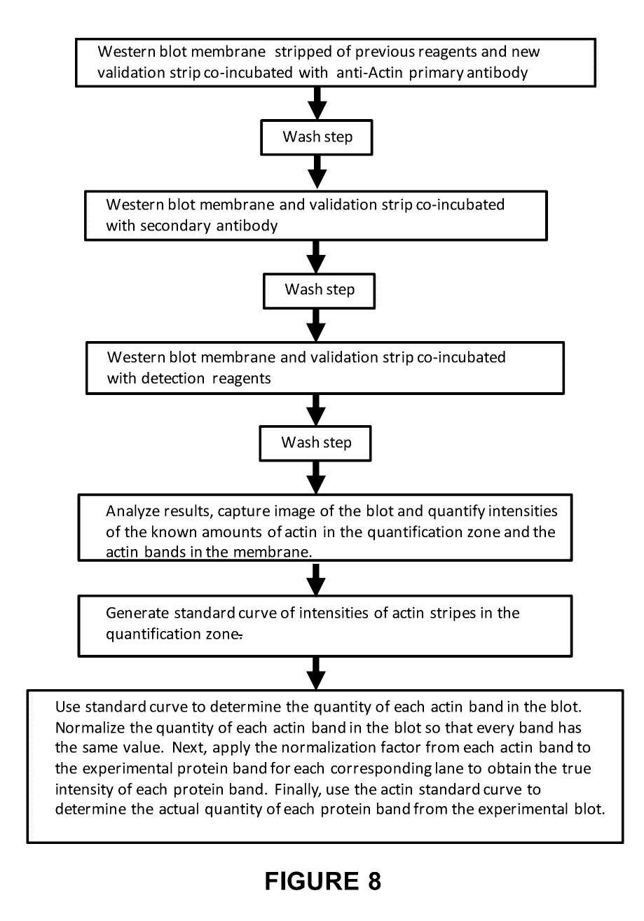

FIG. 8 illustrates a flowchart of reusing a Western blot membrane that has been stripped of the original reagents to re-probe with a different set of primary and secondary antibody specific for a different protein that is present within the fractionated sample proteins immobilized on the membrane. In this example, the flowchart shows the steps involved in the detection of actin “housekeeping” protein within the samples for the purposes of normalizing the sample to sample variability and perform absolute quantification of the protein of interest within the samples. All steps in the process are identical to those described in FIG. 7 except that the primary antibody and corresponding secondary antibody is specific for actin protein. A new V&Q strips is preferably used in this process, however the V&Q strips from the previous steps can be reused as well after performing the stripping steps similar to the membrane. Upon completion of the detection step, the image on the membrane and the strip are digitally captured and once again, using the quantification software, individual actin bands are quantified.

In some embodiments, it is not necessary to strip and re-probe the Western blot and V&Q strip of the disclosure with a new primary antibody. In experiments where, in a given lane of the Western blot, the band corresponding to the housekeeping protein (quantification reagent) and the band corresponding to the target protein of interest are substantially physically separated and do not overlap, both primary antibodies (one primary antibody specific to the housekeeping protein and one primary antibody specific to the target protein of interest) can be added and detected in a single experiment. Substantial physical separation in a Western blot between a housekeeping protein and a target protein of interest may exist where, for example, there is a substantial difference in molecular weight between the housekeeping protein and the target protein of interest. For example, a difference of at least 5-10 kD can be readily resolved in typical Western blot experiments. In general, the resolving power of a Western blot is a function of the percentage of acrylamide used in the Western blot gel, where typical Western blot experiments use 12-15% acrylamide gels. Higher percentage (e.g., 20%) acrylamide gels can resolve, for example, molecular weight differences of 1-2 kD.

In embodiments where the quantification reagent is the same as the target protein of interest being assayed or investigated via the Western blot, no stripping and reprobing steps are necessary. Rather, the entire experiment, including the quantification of the target protein of interest, can be accomplished in a single experiment. When the target protein of interest and the quantification reagent are the same, the amount of target protein of interest in a Western blot can be quantified by: (a) incubating a V&Q strip of the disclosure and a Western blot membrane with an antibody specific for the quantification reagent (which is the same as the target protein of interest being assayed in the Western blot); (b) processing the validation strip and the Western blot membrane to enable detection of the quantification reagent in the validation strip, and to enable detection of the target protein of interest in the Western blot; (c) determining the intensities of the at least two different amounts of quantification reagent in the quantification zone, and determining the intensities of the bands corresponding to the target protein of interest in the Western blot; (d) generating a standard curve correlating the intensities of the quantification reagent in the quantification zone to the amounts of quantification reagent in the quantification zone; and (e) using the standard curve to determine the amount of target protein of interest in each lane from previously determined intensities of protein of interest in each lane.

It should be understood the arrangements and functions described herein are presented for purposes of example only, and that numerous variations are possible. For instance, elements can be added, omitted, combined, distributed, reordered, or otherwise modified.

1. A Western blot process validation strip comprising a strip body, which comprises:
a primary-antibody validation zone comprising a primary-antibody validation reagent, wherein the primary-antibody validation reagent binds specifically to a Western blot primary antibody;
a secondary-antibody validation zone comprising a secondary-antibody validation reagent, wherein the secondary-antibody validation reagent binds specifically to a Western blot secondary antibody; and
a detection validation zone comprising a detection reagent, wherein the detection reagent reacts with a detection substrate.

2. The validation strip of claim 1, further comprising:
a quantification zone comprising at least two different amounts of a quantification reagent.

3. The validation strip of claim 1, wherein the primary-antibody validation reagent is an anti-rabbit antibody, an anti-goat antibody, an anti-sheep antibody, an anti-mouse antibody, or an anti-rat antibody; and wherein the primary-antibody validation reagent is isolated from an organism different from that of the Western blot primary antibody.

4. The validation strip of claim 1, wherein the secondary-antibody validation reagent is isolated from the same organism as that of the Western blot primary antibody.

5. The validation strip of claim 1, wherein the detection validation zone comprises two detection reagents, which are horseradish peroxidase and alkaline phosphatase.

6. The validation strip of claim 1, wherein the quantification reagent is (a) a housekeeping protein selected from the group consisting of GAPDH, beta-actin, and beta-tubulin; or (b) a target protein of interest being assayed in a Western blot.

7. The validation strip of claim 1, wherein the strip body comprises a nitrocellulose or PVDF membrane.

8. The validation strip of claim 1, wherein the primary-antibody validation zone and secondary-antibody validation zone each comprise at least two different amounts of the validation reagent therein.

9. The validation strip of claim 1, wherein the reagents in each zone are laid as stripes in adjacent lines across the width of the strip.

10. A method of validating a Western blot experiment, the method comprising co-processing the Western blot process validation strip of claim 1 along with a Western blot membrane, wherein the Western blot experiment is validated where colored deposits are detected in the primary-antibody validation zone, the secondary-antibody validation zone, and the detection validation zone of the validation strip.

11. A method of quantifying the amount of housekeeping protein in a Western blot, the method comprising:
(a) incubating the validation strip of claim 2 and a Western blot membrane with an antibody specific for the quantification reagent, wherein the quantification reagent is a housekeeping protein;
(b) processing the validation strip and the Western blot membrane to enable detection of the quantification reagent;
(c) determining the intensities of the at least two different amounts of quantification reagent in the quantification zone, and determining the intensities of the bands corresponding to the housekeeping protein in the Western blot;
(d) generating a standard curve correlating the intensities of the quantification reagent in the quantification zone to the amounts of quantification reagent in the quantification zone; and
(e) quantifying the amount of the housekeeping protein in each lane of the the Western blot by correlating the housekeeping protein intensities to housekeeping protein amounts using the standard curve.

12. The method of claim 11, further comprising: in step (c), normalizing the intensities of the bands corresponding to the housekeeping protein to a single housekeeping protein band intensity; or, in step (e), normalizing the amounts of housekeeping protein in each lane of the Western blot to a single amount of housekeeping protein.

13. The method of claim 12, wherein the single housekeeping protein band intensity is the intensity of the highest-intensity housekeeping protein band on the Western blot, and wherein the single amount of housekeeping protein is the highest amount of housekeeping protein in any one lane on the Western blot.

14. The method of claim 12, wherein the single housekeeping protein band intensity is the average of the intensities of the housekeeping proteins bands on the Western blot, and wherein the single amount of housekeeping protein is the average of the amounts of housekeeping protein from all lanes on the Western blot.

15. A method of quantifying the amount of a protein of interest in a Western blot, the method comprising:
(a) incubating the validation strip of claim 2 and a Western blot membrane with an antibody specific for the quantification reagent, wherein the quantification reagent is a housekeeping protein;
(b) processing the validation strip and the Western blot membrane to enable detection of the quantification reagent;
(c) determining the intensities of the at least two different amounts of quantification reagent in the quantification zone, and determining the intensities of the bands corresponding to the housekeeping protein in the Western blot;
(d) generating a standard curve correlating the intensities of the quantification reagent in the quantification zone to the amounts of quantification reagent in the quantification zone; and
(e) using the standard curve to determine the amount of protein of interest in each lane from previously determined intensities of protein of interest in each lane.

16. The method of claim 15, further comprising using a normalization factor for each lane to normalize the previously determined intensities of protein of interest in each lane or to normalize the amounts of protein of interest in each lane,
wherein the normalization factor for a given lane is determined by calculating the factor necessary to normalize the intensity of the housekeeping protein band in that lane to a single housekeeping protein band intensity, or the factor necessary to normalize the amount of housekeeping protein in that lane to a single amount of housekeeping protein.

17. The method of claim 16, wherein the single housekeeping protein band intensity is the intensity of the highest-intensity housekeeping protein band on the Western blot, and wherein the single amount of housekeeping protein is the highest amount of housekeeping protein in any one lane on the Western blot.

18. The method of claim 16, wherein the single housekeeping protein band intensity is the average of the intensities of the housekeeping proteins bands on the Western blot, and wherein the single amount of housekeeping protein is the average of the amounts of housekeeping protein from all lanes on the Western blot.

19. A method of quantifying the amount of a protein of interest in a Western blot, the method comprising:
(a) incubating the validation strip of claim 2 and a Western blot membrane with an antibody specific for the quantification reagent, wherein the quantification reagent is a target protein of interest being assayed in the Western blot;
(b) processing the validation strip and the Western blot membrane to enable detection of the quantification reagent in the validation strip, and to enable detection of the target protein of interest in the Western blot;
(c) determining the intensities of the at least two different amounts of quantification reagent in the quantification zone, and determining the intensities of the bands corresponding to the target protein of interest in the Western blot;
(d) generating a standard curve correlating the intensities of the quantification reagent in the quantification zone to the amounts of quantification reagent in the quantification zone; and
(e) using the standard curve to determine the amount of target protein of interest in each lane from previously determined intensities of protein of interest in each lane.
